(19) United States
(12) Patent Application Publication
(10) Pub. No.: US 2004/0175773 A1
(43) Pub. Date: Sep. 9, 2004

(54) Screening method based on siah-numb interaction

(76) Inventors: Robert Amson, Paris (FR); Adam Telerman, Paris (FR); Moshe Oren, Rehovot (IL)

Correspondence Address: JONES DAY, 51 Louisiana Aveue, N.W, WASHINGTON, DC, 20001-2113, US

(21) Appl. No.: 10/451,887

(22) PCT Filed: Sep. 18, 2001
(86) PCT No.: PCT/FR01/02895

(30) Foreign Application Priority Data
Dec. 26, 2000 (FR) 00/17029

(51) Int. Cl. G01N 33/574

(52) U.S. Cl. 435/7.23

(57) ABSTRACT

This invention encompasses methods of screening the compounds that are involved in the regulation of tumor suppression and apoptosis based on the interaction between Siah and Numb proteins.

The present invention relates to methods for detecting, identifying and/or screening for compounds which are present in the biological chain of p53 and which can be used, in particular, for treating cancers or certain neurodegenerative diseases linked to disturbances in the regulation of tumour suppression and/or apoptosis.

Apoptosis, or cell death, is a complex phenomenon which is regulated by a large number of proteins including the protein p53. This protein interacts with a large number of other proteins and its expression, which induces the phenomena of cell death and tumour reversion, can be correlated with the induction or suppression of the expression of other genes in the cell.

Thus, the inventors of the present invention have demonstrated the presence of genes which are induced and activated during the cascade which leads to tumour suppression and/or apoptosis (TSAP standing for “tumour suppressor activated pathway”) or of genes which are suppressed (TSIP standing for “tumour suppressor inhibited pathway”). These genes constituted, in particular, the subject matter of the patent applications WO 97/22695 or WO 00/08147.

It is important to be able to understand the mechanisms of the p53 cascade clearly in order to be able to generate novel compounds which possess antitumour activity (and which are able, in particular, to induce apoptosis or tumour suppression) or which can be used for treating neurodegenerative diseases. Thus, the inventors of the present application have demonstrated that presenilin 1 (PS1), which has been suggested to play a role in Alzheimer's disease, is identical to the protein TSIP 2 which is described in the application WO 97/22695. In the same way, it is justifiable to search for drugs which can interfere in apoptosis, for the purpose of reducing this phenomenon, and which could be used in neurodegenerative diseases.

The present invention relates to processes for screening for and identifying products which are able to interfere in the p53 cascade and thus induce tumour reversion and/or apoptosis or, conversely, diminish the phenomena of apoptosis.

The present invention is based on the fact that the protein TSAP 3, which is described in patent application WO 97/22695, binds to the protein Numb (as described in the GenBank database under accession number AF015040), which itself exhibits interactions with Mdm2, which is a protein involved in the p53 control processes.

The present invention is also based on the fact that the binding of TSAP 3 (also identified as being protein Siah-1, which can equally well be given this designation or the designation Siah) to Numb induces degradation of the Numb protein by a pathway which is dependent on the ubiquitin proteasome.

Thus, in a first embodiment, the present invention relates to processes for screening for and/or selecting or identifying a compound which interferes with, reduces or inhibits the binding of Siah to Numb, which processes exhibit the steps of:

a) bringing the said compound into contact with a mammalian cell which is expressing the Siah and Numb proteins;

b) identifying the increase in the quantity of Numb protein in the said mammalian cell as compared with a control cell with which the said compound has not been brought into contact.

In a second embodiment, the invention is directed towards a process for screening for, selecting or identifying a compound which interferes with, reduces or inhibits the binding of Siah to Numb, which process exhibits the steps of:

a) bringing the said compound into contact with a system which enables the binding between Siah and Numb to be determined in vitro;

b) identifying the decrease in and/or inhibition of the binding between Siah and Numb.

In another embodiment, the teachings of the present invention will make it possible to identify compounds which, on binding to Numb, possibly in place of Siah, will induce a degradation of Numb which is greater than the degradation induced by the Siah protein. As a consequence, the decrease in the quantity of Numb will involve an increase in the quantity of p53 which is present and an increase in tumour suppression and/or cell death (apoptosis). Consequently, the invention is directed towards a process for screening for, selecting and/or identifying a compound for increasing tumour suppression and/or cell death (apoptosis), which process exhibits the steps of:

a) bringing the said compound into contact with a mammalian cell which is expressing the Siah and Numb proteins;

b) identifying the decrease in the quantity of Numb protein in the said mammalian cell as compared with a control cell with which the said compound has not been brought into contact.

Conversely, the present invention also relates, in particular, to a process for screening for, selecting or identifying a compound for decreasing and/or inhibiting tumour suppression and/or cell death (apoptosis), which process exhibits the steps of:

a) bringing compounds into contact with a system which enables the binding between Siah and Numb to be determined in vitro;

b) identifying the compounds which induce the decrease in and/or inhibition of the binding between Siah and Numb;

c) bringing the compounds identified in step b) into contact with a mammalian cell which is expressing the Siah and Numb proteins;

d) identifying the increase in the quantity of Numb protein in the said mammalian cell as compared with a control cell with which the said compound has not been brought into contact.

In order to determine compounds which increase tumour suppression and/or cell death (apoptosis), it is also possible to combine different processes according to the invention; in this way, it is possible, in particular, to obtain a process for screening for, selecting or identifying compounds whose function is to increase tumour suppression and/or cell death (apoptosis), which process exhibits the steps of:

a) bringing the said compound into contact with a system which enables the binding between Siah and Numb to be determined in vitro;

b) identifying the compounds which induce the decrease in and/or inhibition of the binding between Siah and Numb;

c) bringing the compounds selected in step b) into contact with a mammalian cell which is expressing the Siah and Numb proteins;

d) identifying the decrease in the quantity of Numb protein in the said mammalian cell as compared with a control cell with which the said compound has not been brought into contact.

A process of this nature is therefore also part of the subject matter of the present invention.

The present invention therefore makes use of the fact that the Siah and Numb proteins can bind to each other. It is therefore worthwhile to identify the domains of each protein which are in fact in contact with the other protein. This is because this ought to make it possible to be able to use the peptides which have thus been identified as baits or agonists of the complete proteins. This can, in, this way, make it possible to specify compounds which will interfere in the Siah-Numb binding and which will be able either to induce tumour suppression and/or apoptosis (in particular if these peptides have an effect on the degradation of Numb (Siah-derived peptides)) or, conversely, to decrease these phenomena (in particular if these peptides prevent the binding of Siah to Numb (whether they are bait peptides derived from Numb or whether they are derived from Siah prevent the binding of this protein to Numb, in particular by competition).

The present invention therefore relates, in particular, to a process for identifying a region of Siah which is active in increasing tumour suppression and/or apoptosis, which process comprises the steps of:

a) bringing peptides derived from the Siah protein into contact with a mammalian cell which is expressing the Numb protein;

b) identifying the decrease in the quantity of Numb protein in the said mammalian cell by comparison with the quantity of Numb protein which is present in a control mammalian cell which is expressing Siah and Numb proteins.

A “region of Siah” is understood, in particular, as meaning peptides having a primary sequence which is derived from the primary sequence of the Siah protein.

The present invention is therefore also directed towards a process for identifying a region of Siah which is active in decreasing and/or inhibiting tumour suppression and/or apoptosis, which process comprises the steps of:

a) bringing peptides derived from the Siah protein into contact with a mammalian cell which is expressing the Numb protein;

b) identifying the increase in the quantity of Numb protein in the said mammalian cell by comparison with the quantity of Numb protein which is present in a control mammalian cell which is expressing the Siah and Numb proteins.

More generally, the present invention makes it possible to identify regions of Siah which are involved in the binding with the Numb protein by means of a process which comprises the steps of:

a) bringing peptides derived from the Siah protein into contact [lacuna] in a system which enables the binding between Siah and Numb to be determined in vitro;

b) identifying the peptides which bring about the decrease in the binding between Siah and Numb in the said system.

It is obvious that the present invention also makes it possible to determine the regions of Numb which are involved in the binding with Siah, in accordance with processes which are similar to the previously described processes that these regions can, in particular, be used as baits when it is desired to decrease tumour suppression and/or apoptosis.

The present invention therefore makes it possible to identify products which can interact with the Siah-Numb binding and which can therefore be of significance in the regulation of apoptosis and/or tumour suppression. Nonetheless, it is possible that, in order to be able to be used for a therapeutic treatment, in particular for treating cancer or neurodegenerative diseases, these products may need to be optimized in order to have a higher activity and/or a lower toxicity.

Thus, the development of a drug is frequently effected in accordance with the following principle:

using an appropriate method to screen for compounds which possess a desired activity,

selecting the compounds which fit the “schedule of conditions” (in this case, an action on the phenomena of apoptosis and tumour suppression),

determining the structure (in particular the sequence (where appropriate tertiary) if the compounds are peptides, or the formula and backbone if the compounds are chemical compounds) of the selected compounds,

optimizing the selected compounds by modifying the structure (for example by changing the stereochemical conformation (for example changing the amino acids in a peptide from L to D), adding substituents to the peptide or chemical backbones, in particular by attaching residues to the backbone, and modifying the peptides (see, in particular, Gante, “Peptidomimetics”, in Angewandte Chemie-International Edition Engl. 1994, 33, 1699-1720),

testing and screening the compounds which have thus been obtained in appropriate models which are frequently models which are those which are closest to the pathology being studied. At this stage, use is frequently made, in particular, of animal models, in general rodent (mice, rats, etc.) models or dog models, or even primate models.

Examples of animal models which can be used in the case of cancer are models which are based on immunosuppressed mice (for example scid/scid mice), which are injected (in particular subcutaneously) with tumour cells which will lead to the development of tumours. The efficacy of the potentially antineoplastic compounds is studied, for example, by measuring the sizes of the tumours which are formed.

In order to study neurodegenerative diseases, it is possible to use the model described by Amson et al. (2000, Proc. Natl. Acad. Sci. USA, 97, 5346-50), which consists of p53-deficient mice, or the model described in Chen et al., Janus et al., and Morgan et al. (2000, Nature, 408 pp. 975-985).

Thus, the present invention relates, in particular, to making it possible to identify compounds which could be used for treating cancer on the basis that they possess an activity in increasing tumour suppression and/or apoptosis. A part of the subject-matter of the present invention is therefore a process which comprises the steps of:

a) implementing a process according to the invention which makes it possible to identify compounds which possess some degree of activity in increasing tumour suppression and/or apoptosis,

b) modifying the product selected in step a),

c) testing the product modified in step b), in in vitro and/or in vivo processes, in models which are relevant to tumour suppression and/or apoptosis,

d) identifying the product which makes it possible to obtain a tumour suppression and/or apoptosis activity which is greater than the activity obtained in the case of the product selected in step a).

Step d) can be replaced by a step d′), which would be:

d′) identifying the product which makes it possible to obtain the sought-after biological effect with a lower toxicity in an animal model (when one of the models used in step c) is an in vivo model).

When the tests are carried out on in vitro models of apoptosis or tumour suppression, it is possible, for example, to use the K256/KS model described by Tellerman et al. (1993, Proc. Natl. Acad. Sci. USA, 90, 8702-6). It is also possible to use the M1-LTR cells described by Amson et al. (1996, Proc. Natl. Acad. SCI; USA, 93, 3953-7) or the U937/US3-US4 cells described by Nemani et al. (1996, Proc. Natl. Acad. Sci. USA, 93, 9039-42).

The in vivo tests can be carried out by injecting these cells animals, in particular immunosuppressed mice, and by studying the effects of the different compounds being tested.

The skilled person will know how to define the necessary conditions and thresholds for identifying a product which can be used as a drug, in accordance with the regulatory requirements (in particular as regards toxicology) and in relation to the benefit provided by the product which has thus been identified.

Similarly, the invention also relates to the processes for optimizing products which counteract tumour suppression and/or apoptosis, which products have been identified by the processes which are described above, and which make it possible to identify products which can be used as drugs.

Thus, the invention also relates to a process for identifying a product which is active in decreasing and/or inhibiting tumour suppression and/or apoptosis, characterized in that it comprises the steps of:

a) implementing a process according to the invention, making it possible to identify compounds which possess some degree of activity in decreasing tumour suppression and/or apoptosis,

b) modifying the product selected in step a), in particular by attaching residues to the chemical backbone,

c) testing the product modified in step b) in in vitro and/or in vivo processes in models which are relevant to tumour suppression and/or apoptosis,

d) identifying the product which makes it possible to obtain a tumour suppression and/or apoptosis activity which is reduced as compared with the activity which is obtained in the case of the product selected in step a).

Step d) can be replaced by a step d′), which would be:

d′) identifying the product which makes it possible to obtain the sought-after biological effect with less toxicity in an animal model (when one of the models used in step c) is in vivo).

Thus, it is a matter, once again, of being able to obtain the product which exhibits the best (biological activity and clinical effect)/(potential risks of use) ratio.

The parameters which are to be brought into play for obtaining these results are all known and within the capacity of the skilled person who wishes to develop novel drugs and can be found, for example, in the directives of the bodies such as the Agence du Medicament [Drug Agency], the European Commission or the Federal Drug Agency.

Implementation of the processes according to the present invention requires models which enable the binding between Siah and Numb to be determined and also an easy measurement of the increase or decrease in the quantity of Numb protein in a eukaryotic cell.

The quantity of Numb protein can be studied by Western blotting, with visualization being effected by using an antibody which is directed against the said protein. Such antibodies can, in particular, be obtained from the Weizmann Institute of Science, Rhovot, Israel, under reference WIS 3465, or from Transduction Laboratories (reference N80220).

In order to implement the processes according to the invention which require studying, and screening of, mammalian cells, it can be advantageous to overexpress one or other of the Siah and Numb proteins in the said cells, or to overexpress both the proteins together.

Thus, it is easier to be able to study the variations in the quantity of Numb protein by, in particular, comparing cells on which the compounds of interest are being tested with the same cells to which the compounds which it is desired to screen are not being added, and cells which express Numb without expressing Siah (positive controls).

It is understood that the expression of the two proteins Siah and Numb can be increased by introducing the genes (in particular the cDNAs) encoding these two proteins into cells, with these genes either being located on vectors or introduced into the chromosome.

When episomal expression is selected, the said mammalian cell is transfected with at least one vector which is selected from a vector carrying a DNA fragment encoding Siah, a vector carrying a DNA fragment encoding Numb and a vector carrying a DNA fragment encoding Siah and a DNA fragment encoding Numb. Thus, the same vector can express both the proteins; alternatively, it is possible to introduce two vectors.

It is possible to use vectors which enable the Siah and Numb proteins to be readily expressed and purified, for example in prokaryotic cells (E. coli, B. subtiis, etc.) or eukaryotic cells (yeasts such as Saccharomyces, Kluyveromiyces, etc., mammalian cells (HeLa, Cos, Hep-2, etc.) or insect cells (using a Baculovirus system) . Thus, it can be advantageous for the proteins to possess a label at their N-terminal or C-terminal end in order to facilitate purification. A histidine or GST label is selected, in particular. These methods are well known to the skilled person, who finds the appropriate plasmids in the catalogues of companies such Stratagene.

When the processes according to the invention are implemented on in vitro models for studying the binding between Numb and Siah, there are several ways of proceeding.

The following protocol can be used:

expressing and purifying the Siah and Numb proteins in, for example, prokaryotic cells (E. coli, B. subtiis, etc.) or eukaryotic cells (yeasts such as Saccharomyces, Kluyveromyces, etc., mammalian cells (HeLa, Cos, Hep-2, etc.) or insect cells (using a Baculovirus system). It can be advantageous for the proteins to possess a label at their N-terminal or C-terminal end in order to facilitate purification. A histidine or GST label is selected, in particular. These methods are well known to a skilled person, who finds the appropriate plasmids in the catalogues of companies such Stratagene;

binding the proteins to suitable beads. When a GST label is used, the proteins which are expressed are bound to sepharose beads which are presenting glutathione;

preparing proteins by translation in vitro. This can easily be effected using commercial vectors (for example available from Promega) which make it possible to clone the cDNAs under the control of well known promoters (T7 or T3), and to use the appropriate RNA polymerases for producing the RNAs and then to express the proteins in vitro, employing disposable kits and following the manufacturer's instructions;

coprecipitating the proteins by adding the proteins obtained by in vitro translation to the sepharose-glutathione beads to which the proteins which are fused with the GST are attached. After an adequate contact time, the beads are washed, and a SDS-PAGE gel electrophoresis analysis is carried out, followed by autoradiography. The appearance of the bands which correspond to two proteins demonstrates clearly that these proteins are bound together.

The use of appropriate controls thus makes it possible to define the decrease in and/or inhibition of the binding between Siah and Numb by comparing the quantities of protein which are released after adding the compound being tested during the coprecipitation step with the quantities of protein released in the controls.

It is also possible to study the binding of the proteins by using the FRET (fluorescence resonance energy transfer) system, which consists in labelling each of the proteins with an appropriate residue, with the binding of the two proteins inducing a reaction between each of the two residues and emission of a readily detectable fluorescence.

The present invention also relates to the compounds which can be obtained by a process according to the invention, in particular the compounds which are active in increasing tumour suppression and/or apoptosis, those which are active in inhibiting Siah-Numb binding and those which are active in decreasing and/or inhibiting tumour suppression and/or apoptosis.

The present invention also relates to the peptide sequences corresponding to a region of Siah which interacts with the Numb protein, which sequences can be identified, in particular, by a process according to the invention.

The invention also relates to the peptide sequences which correspond to a region of Numb which interacts with the Siah protein, which sequences can be identified, in particular, by a process according to the invention, with the process which enables the peptide sequences of Siah which interact with Numb to be identified being able to be adapted for determining the peptide sequences of Numb which interact with Siah, in particular by adapting the in vitro protocol which was elaborated on above.

The invention also relates to the nucleotide sequences which encode the peptide sequences which have thus been identified.

It is clear that the term peptide sequence or nucleic acid sequence or nucleotide sequence (with these two latter terms being used interchangeably) represent isolated sequences, that is sequences which are out of their natural state and which can be modified, in particular, by replacing their base units with unnatural units or by modifying the bonds between the base units (for example, phosphorothioates (nucleic acid) or peptide nucleic acids).

It is part of the subject matter of the invention to enable compounds which interfere with the binding of Siah and Numb to be identified, with some of these compounds being able, in particular, to induce effects on the p53 cascade. Thus, the compounds according to the invention, the peptide sequences according to the invention, or the nucleotide sequences according to the invention, are, as drugs, also part of the subject matter of the invention.

A compound which is identified by a method according to the invention can be a compound having a chemical structure, a lipid, a sugar, a protein, a peptide, a protein-lipid, protein-sugar, peptide-lipid or peptide-sugar hybrid compound, or a protein or a peptide onto which chemical branchings have been added.

The chemical compounds which are envisaged may contain one or more aromatic or non-aromatic rings as well as several residues of any nature (in particular lower alkyl, that is possessing between 1 to 6 carbon atoms).

These compounds, nucleic acid sequences and peptide sequences can thus be used for preparing a drug which is intended, in particular, for treating cancer or a neurodegenerative disease, depending on the pro- or anti-apoptosis/tumour reversion effect.

The inventors of the present application have, for the first time, demonstrated the fact that the Siah protein binds to the Numb protein. Thus, the present invention also relates to a complex which consists of a Siah protein and a Numb protein.

The present invention also relates to a process for inhibiting the binding of Siah to Numb in a cell, which process comprises the step of:

a) bringing the said cell into contact with a compound which has been identified by a process according to the invention and which inhibits the Siah-Numb binding.

The compound which is thus envisaged can -also be a “bait” peptide which is derived from the Siah protein or the Numb protein. The process can be implemented in vitro or in vivo.

The present invention is also directed towards a method for treating a cancer, which method is characterized in that a compound which has been identified according to the present invention, and which increases apoptosis and/or tumour reversion, is administered to a patient.

A method for treating a neurodegenerative disease, which method consists in administering, to a patient, a compound which is in accordance with the present invention and which decreases or inhibits apoptosis, is also part of the subject matter of the present invention.

As made clear and demonstrated in the examples, besides binding to the Numb protein, the Siah protein also induces its degradation by way of the ubiquitin-linked proteasome pathway. It is noteworthy that the Numb protein is known for binding to the Mdm2 protein, the presence of which induces negative regulation of p53. It is furthermore known that the expression of Siah in cells induces apoptosis phenomena, as the inventors have previously demonstrated. Thus, the present invention also provides a process for inhibiting the decrease in p53, which is linked, in particular, to Mdm2 via Numb, in a cell, which process comprises the step of:

a) bringing the said cell into contact with a compound according to the invention which increases apoptosis and/or tumour reversion.

Conversely, it is possible to augment the decrease in the quantity of p53 protein in a cell, in particular on account of the interactions between Mdm2 and Numb, which process comprises the step of:

a) bringing the said cell into contact with a compound according to the invention which inhibits or decreases apoptosis and/or tumour suppression.

These two processes can be implemented in vitro or in vivo, in particular within the context of a therapeutic treatment.

### LEGENDS TO THE FIGURES

FIG. 1: Analysis of the in vitro interaction between Siah-1 and Numb. FIG. 1.A. Fusion proteins GST-68 (with protein 68 being the Leu 82-Glu 314 fragment of BNIP 2) (columns 1 and 2) and GST-Numb (columns 3 and 5), seen using Coomassie Blue. FIG. 1.B. Autoradiography of the in vitro translation products of the proteins AIP1 (column a) and Siah-1 (column b), with these products being labelled with ³⁵S-methionines. Columns 1 to 4 illustrate the precipitation of AIP1 (1 and 3) or Siah-1 (2 and 4) with the products shown by the GST fusion proteins.

FIG. 2: Analysis of the effect of the in vivo interaction between Siah and Numb on the levels at which the proteins are expressed. FIG. 2.A. 293T cells were cotransfected with an empty expression vector (column 1) or expressing Numb (column 2), Siah-1 (column 3), Numb and Siah-1 (column 4), Numb and Mdm2 (column 5), and Numb, Siah-1 and Mdm2 (column 6). 48 hours after transfection, a Western blot analysis was carried out using an anti-Numb antibody. A control was also performed using an anti-GFP (green fluorescent protein) antibody as a loading standard on the gel. FIG. 2.B. The Western blot was analysed as previously described in the absence or presence of an inhibitor of the ubiquitin proteasome (MG 132). Without MG 132, the quantity of Numb protein decreases in the presence of Siah-1, with or without Mdm2 (lanes 2, 3 and 4). With MG 132, the quantity of Numb protein remains constant (lanes 6, 7 and 8).

FIG. 3: Analysis of the in vivo and in vitro interaction of Siah-1 and Numb

A. Summary of the yeast two-hybrid data. Siah-1 and Numb interact by means of an interaction assay in a yeast two-hybrid. The LexA-Siah-1 and B42-Numb interactions were tested either by growth or by β-galactosidase activity. In the presence of galactose (Gal), the targets containing B42, labelled for activation, are expressed whereas they are suppressed on glucose (Glu). Yeasts harbouring combinations of target plasmids and bait plasmids as negative controls, i.e. LexA-RFHM1/B42-Cdi3, Lex A-Siah-1/B42-Cdi3, LexA-RFHM1/B42-Numb, or positive control combinations, i.e. LexA-RFHM12/Cdi3 and LexA-Siah-1/B42-Siah-1, were tested in parallel. As expected, the yeasts harbouring the target and bait plasmids as positive controls exhibited growth and were β-gal⁺ in the presence of galactose but not of glucose. Furthermore, the yeasts harbouring LexA-Siah-1 and B42-Numb also exhibited strong galactose-dependent growth and β-gal activity.

B. In vitro interaction of GST-Siah-1 and radiolabelled Numb. Numb and the negative control AIP1 were generated by in vitro translation (IVT) in a rabbit reticulocyte lysate in the presence of methionine and cysteine which were both labelled with ³⁵S. Equal quantities of radiolabelled products were incubated either with GST-Siah-1 or GST-NKTR fusion proteins, as negative control, with these proteins being captured on glutathione beads. The radiolabelled proteins were eluted in a protein sample buffer, resolved, under reducing conditions (0.7 mM 2-β-mercaptoethanbl), by electrophoresis on a sodium dodecyl sulphate-poly-acrylamide (SDS-PAGE) gel (10%), and visualized by autoradiography.

C. Reciprocal combination between GST-Numb and Siah-1. AIP1 or Siah-1, derived from an in vitro translation (IVT), were generated and GST pull-down assays were carried out using either GST-NKTR or GST-Numb, as described above. The representation of the contribution of the indicated GST is shown on the left-hand panels in B and C. It is to be noted that GST-Numb is partially degraded.

D. In vivo interaction of Flag-Numb and Siah-1. A construction designated Siah-1m, carrying 3 false-sense mutations (Cys→Ser) at amino acid positions 129, 131 and 135 in Siah-1, was generated so as to give rise to a more stable protein. 293T cells were cotransfected transiently with either Flag-AIP1 and Siah-1m or Flag-Numb and Siah-1m using the calcium phosphate precipitation method. The levels at which Flag-AIP1 and Flag-Numb were expressed in the total cell lysates (TLS) were determined by means of immunolabelling using an anti-flag antibody (D, left-hand panel). The Siah-1-Numb association extensions were determined by immunoprecipitating Flag-Numb with a Flag antibody, followed by immunolabelling using an antiserum directed against Siah-1 (D, right-hand panel). It is to be noted that the monomeric form of Siah-1 migrates as a peptide of approximately 32 kDa in size. The protein band of approximately 60 kDa in size in the Siah-1 total lysate probably represents Siah-1 dimers.

E. Endogenous interaction of Siah-1 and Numb in U937 cells and Jurkat cells. 2.5×10⁸ cells were chemically linked by crosslinking with the reducible DTBP linker. The cell-derived lysates were incubated overnight with either an anti-Ig which pairs the species, as a negative control, or an anti-Numb antibody, followed by incubation with G-agarose-proteins. The immune complexes were resolved in a 12% SDS-PAGE gel and then labelled with an anti-Siah-1 antibody. It is to be noted that the anti-Numb antibody coimmunoprecipitates the 32 kDa monomeric form of Siah-1 both in the U937 cells and in the Jurkat cells (E, right-hand panel). The right-hand panel shows the endogenous expression of Numb in the two cell lysates (TL).

FIG. 4: Mapping of the Siah-1 and Numb interaction domains.

A.-C. GST pull down assays were carried out by incubating the indicated radiolabelled fragments of Siah-1 (Δ1-10), which were derived from an in vitro translation (IVT), or the negative control AIP1, with GST-NKTR (data not shown) or several (ΔA-C) Numb GST fusion proteins. Samples were resolved as described above. It is to be noted that GST-Numb (second panel) interacts with a minimal fragment of Siah-1, i.e. Δ10 composed of amino acids 180-211.

B. Representation of the contribution of GST.

C. and D. Diagram depicting the Siah-1 and Numb deletion mutants, respectively. The black bars shown in the full-length Numb pinpoint motifs containing SH3.

(*) in C. indicates the location of the three false-sense mutations.

FIG. 5: Effects of the overexpression of Siah-1 on the degradation of Numb and on ubiquitination.

A.-C. Siah-1 induces degradation of Numb in vivo.

A. 293T cells were cotransfected transiently with the indicated plasmids, using the method of calcium phosphate precipitation. 48 hours after the transfection, the total cell lysates were prepared and resolved by means of an SDS-PAGE analysis followed by antibodies. In order to verify that the protein samples had been loaded to equivalent extents, the membranes were stripped and reprobed with an anti-tubulin antibody (A., lower panel).

B. The stable-state levels of Numb were analysed by means of immunolabelling analysis, either in the presence or in the absence of the proteasome inhibitor MG132 (50 μM), which inhibitor was added 3 hours before harvesting the cells.

C. In vivo ubiquitination of Numb by Siah-1. The 293T cells were transfected transiently with plasmids encoding flag-Numb, HA-polyubiquitin and varying quantities of Siah-1 (10-30 μg). 24 hours after the transfection, the cells were incubated with an anti-flag antibody. The immunoprecipitates were analysed for ubiquitination by immunolabelling with an anti-HA. It is to be noted that the MG132 (50 μM) was added to 293T transfectants 6 hours before the harvesting with the aim of preventing the degradation of Numb. HC pinpoints the heavy chain of the immunoglobulin (C, upper panel). Mdm2 was used as a positive control reference point. The middle panel shows the levels of flag-Numb expression as visualized using the anti-Numb antibody. The lower panel shows the overall outline of the ubiquitination as detected by the anti-HA using increasing quantities of Siah-1 or Mdm2.

FIG. 6: In vivo effects of Siah-1 on the endogenous degradation of Numb

A.-D. The potentiation of Siah-1 which was induced by p53 promotes decreased levels of endogenous Numb.

A. 24 hours after having subjected the LTR6 cells to 32° C., which activates p53, the cell lysates were prepared and analysed with regard to the levels at which endogenous Siah-1 (A. lower panel) and endogenous Numb (upper panel) were expressed. It is to be noted that the increased levels of Siah-1 proteins are concomitant with a decrease in the expression of Numb. B. Analysis of LTR6 cells which are transfected stably with an anti-sense Siah-1. Even at 37° C., the anti-sense Siah-1 inhibited the expression of the 32 kDa Siah-1 protein (B. middle panel, band 2). The increase in Siah-1 following p53 activation (B. middle panel, band 3) was significantly inhibited in the cells which were transfected with an anti-sense construct. This reduced the expression of Siah-1 which resulted from the increased level of the Numb protein (B. upper panel, band 4).

C. The left-hand panel shows a FACS analysis of annexin-v-positive cells following activation of p53 in LTR6 cells which were either transfected with a vector on its own or with the anti-sense Siah-1. The significant reduction in p53-induced apoptosis with the anti-sense Siah-1 is to be noted.

C. Right-hand panel, the LTR6 total cell lysates were prepared and resolved by an SDS-PAGE analysis followed by an immunolabelling using either an anti-PARP antibody or an anti-tubulin antibody.

D. U937 cells, or stable U937 transfectants expressing either the control vector or Siah-1, were analysed for endogenous Numb in the total cell lysates. In addition, the clone US4, which is derived from U937 cells, was also analysed for the endogenous levels of the Siah-1 and Numb proteins. Lysates were probed with either the anti-Numb antibodies (D. top panel) or the anti-Siah-1 antibodies (D. central panel), or the anti-tubulin antibodies (D. lower panel). The reduction in the levels of Numb protein in Siah-1 which are overexpressing the U937 cells is to be noted.

FIG. 7: In vivo effects of Siah-1 overexpression on Notch activity.

Redistribution of endogenous Notch1 in U937 cells which are overexpressing Siah-1.

A. Stable U937 transfectants which were expressing either the control vector (panels 1 to 3) or Siah-1 (panels 4 to 7) were analysed using a confocal microscope for the endogenous immunofluorescence of Notch1. The cells were labelled with an antibody which recognized the cytoplasmic portion of human Notch1, followed by an FITC-conjugated secondary antibody. The Notch immunofluorescence analysis is related either to low magnification (panels 1 and 4) or to high magnification (panels 2, 3, 5, 6 and 7). Note the border outline-type labelling of Notch1 in the U937 cells harbouring the control vector as compared with the nuclear and cytoplasmic labelling in the cells which were overexpressing Siah-1. The labelling with propidium iodide was included in order to better visualize the nuclear labelling of Notch1 (panels 3, 6 and 7). The presence of Notch1 in the nucleus was confirmed by the Z-plane image (panel 7). The bar represents a length of 5 μm.

B. Panels 8 to 10 show the nuclear translocation of Notch1 which was induced by EDTA in the U937 cells harbouring the control vector (panels 8 to 10). Cells were incubated in 10 mM EDTA containing PBS at 37° C. for 30 min, and then washed and labelled with anti-Notch1 (intracellular) antibodies either immediately (T=0) or 30 min (T=30) after removing the EDTA. The cells were counterlabelled with propidium iodide in order to visualize the nucleus. Note the presence of positive Notch1 labelling in the nucleus at T=30. Panel 8 shows the untreated U937 cells.

C. Notch1 activity. MCF-7 cells and stable MCF-7 transfectants which were overexpressing Siah-1 were transfected transiently with an NICD (pGA981-6) reporter construct and were monitored for NICD activity at 48 hours after infection. Note the augmented endogenous NICD reporter activity in the MCF-7 cells which were stably transfected with Siah-1. This increased NICD activity was effectively inhibited by a cotransfection with Numb.

### EXAMPLES

### Example 1

### Expressing the GST Fusion Proteins

Preparation of a preculture from an isolated colony of B121 (DE3), which is transformed with the plasmid pGEX-P-1-Siah or pGEX-P-1-Numb, at 37° C. in an SB medium containing 100 μg of ampicillin/ml.

The plasmid PGEX-P-1 can be obtained from Amersham Pharmacia Biotech AB.

The proteins are the human proteins encoded by the complementary cDNAs corresponding to the sequences SEQ ID No. 1 (Siah) and SEQ ID No. 2 (Numb). The references for the different proteins in GenBank are HSU76247, in the case of human Siah, and AF015040, in the case of human Numb.

On the following day, 250 ml of SB+Amp are inoculated with 5 ml of the preculture.

Growth is carried out at 37° C. in the case of GST-Numb and 28° C. in the case of GST-Siah up to an optical density of between 0.5 and 0.7.

0.1 mM IPTG is added in order to induce synthesis of the proteins.

Growth is carried out at 37° C. in the case of GST-Numb and 28° C. in the case of GST-Siah for 1 h 30 min (GST-Numb) and 1 h (GST-Siah).

The cultures are centrifuged at 3 000 rpm for 10 min (1 800 g, at 4° C.).

The precipitate is resuspended in 10 ml of A NP40 buffer (NP40, 1%; Tris, pH 7.4, 10 mM; NaCl, 150 mM; EDTA, 1 mM; glycerol, 10%; DTT, 1 MM; Aprotinin, 2 μg/ml; Leupeptin, 2 μg/ml; Pepstatin, 2 μg/ml; AEBSF, 1 mM).

Cells are sonicated 3 times for 15 s at strength 50 and on ice.

The mixture is centrifuged at 12 000 rpm for 10 min (18 000 g at 4° C.).

The supernatant is stored at −80° C.

### Example 2

### Binding to the sepharose-glutathione Beads

The whole of the preparation of the beads, and the incubations, are carried out at 4° C.

2 ml of supernatant are added to 200 μl of beads (prepared after 3 rinsings in PBS and 1 rinsing in A NP40 buffer followed by resuspension at a concentration of 50% (weight per volume) in A NP40 buffer, with centrifugation at 3 000 rpm on each occasion).

Glutathione-sepharose 4B beads, which are supplied by Amersham Pharmacia Biotech AB under reference 17.0756.01, are used.

These are shaken gently at 4° C. for at least 1 hour.

The beads are rinsed 3 times in A NP40 buffer which does not contain any protease inhibitor.

The beads are resuspended in 1 ml of A NP40 buffer containing protease inhibitor.

For the SDS-PAGE electrophoresis analysis, from 20 to 40 μl of the resuspended beads are taken and centrifuged for 5 min; the supernatant is removed and the beads are resuspended in 10 μl of X loading buffer and heated at 97° C. for 7 min. The gel is loaded and analysed after visualization with Coomassie Blue in order to standardize the quantity of fusion proteins to be used.

### Example 3

### In vitro Translation

The TNT Coupled Reticulocyte Lysate System kit supplied by Promega was used, together with T7 RNA polymerase or T3 RNA polymerase, depending on the vector employed, for translating and expressing the proteins (T7 in the case of AIPI and Siah1, and T3 in the case of Numb). The kit was used in accordance with the manufacturer's instructions (reference L4610).

The proteins incorporated ³⁵S-methionine (Amersham Pharmacia).

The products which were obtained in vitro were analysed by SDA-PAGE electrophoresis.

After electrophoresis, the gel is placed in fixing buffer (5% methanol, 15% acetic acid, 80% water) for half an hour and the signal is amplified by immersing the gel in the Amplify product supplied by Amersham Pharmacia (reference NAMP 100).

A Kodak Biomax MR film is then exposed on the dried gel for a period varying from one hour to one week and then developed.

### Example 4

### Coprecipitating the Proteins

After the quantities had been standardized, 30 μl of the sepharose-glutathione beads, coupled to the GST fusion proteins, were rinsed in buffer B (NP40, 1%, TrisHCl, 50 mM, NaCl, 150 mM, leupeptin, 2 μl/ml, aprotinin, 1%, ABESF, 1 mM).

From 5 to 10 μl of the in vitro translation product, as obtained in Example 3, are then added depending on the quantity of the product which is observed by autoradiography.

After overnight contact, the beads were rinsed 10 times with A NP40 buffer lacking antiproteases.

Analysis is carried out by SDS-PAGE and auto-radiography.

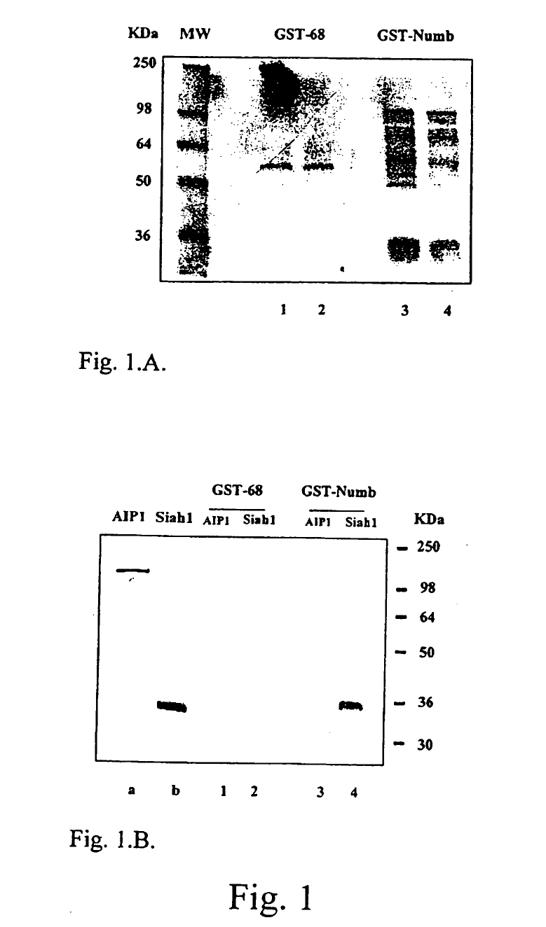

FIG. 1B therefore clearly shows that the Siah protein obtained by in vitro translation binds to the GST-Numb fusion protein.

It is possible to show, in the same way, that the Numb protein obtained by in vitro translation binds to the GST-Siah fusion protein (not shown).

### Example 5

### Cells and Transient Transfections

The 293T human embryonic kidney cells (ATCC reference: CRL 1573) are maintained in DMEM, which is supplemented with 10% foetal calf serum (FCS), in an atmosphere containing 5% CO₂. The cells underwent a new passage on the day prior to the transfection.

The 293T cells were transfected by the calcium phosphate procedure in DMEM plus 10% FCS and in the presence of 20 μM chloroquine. Eight hours after the transfection, the transfection medium was replaced with DMEM plus 10% FCS. The cells were harvested 48 hours after the transfection.

For the tests using MG132, the transfected cells were treated, at 37° C. for 6 hours, with 50 μM MG132 (Calbiochem-Novabiochem) in DMSO, or with DMSO on its own as a control. The plasmids expressing the human Numb protein, murine Mdm2 and GFP were pcDNA3 plasmids obtained from Invitrogen, San Diego, Calif., USA.

The Siah-1 protein was obtained from the complementary DNA which was subcloned into the plasmid pBKRSV (Stratagene, La Jolla, Calif., USA).

### Example 6

### Western Blotting

48 hours after transfecting the 293T cells with the plasmids, the cells were lysed in a TBS-Triton lysis buffer (Tris-buffered saline [TBS], [pH 8.0]; 1% Triton X-100, 10 mg of phenylmethylsulphonyl fluoride/ml; aprotinin, 5 mg/ml, leupeptin, 5 mg/ml).

The cell lysates were separated by SDS-PAGE gel electrophoresis and transferred to a nitrocellulose membrane (Biorad), with this being followed by a Western blot analysis using the Numb polyclonal antibody (Weizmann Institute, Ref. 3465, or reference N80220 from Transduction Laboratories) and the anti-GFP monoclonal antibody (Boehringer, Ref. 1814460) in order to verify standardization of the quantities which were loaded onto the gel. The signals were detected using a secondary antibody coupled to a peroxidase and to electrochemoluminescent reagents supplied by Amersham Pharmacia Biotech AB.

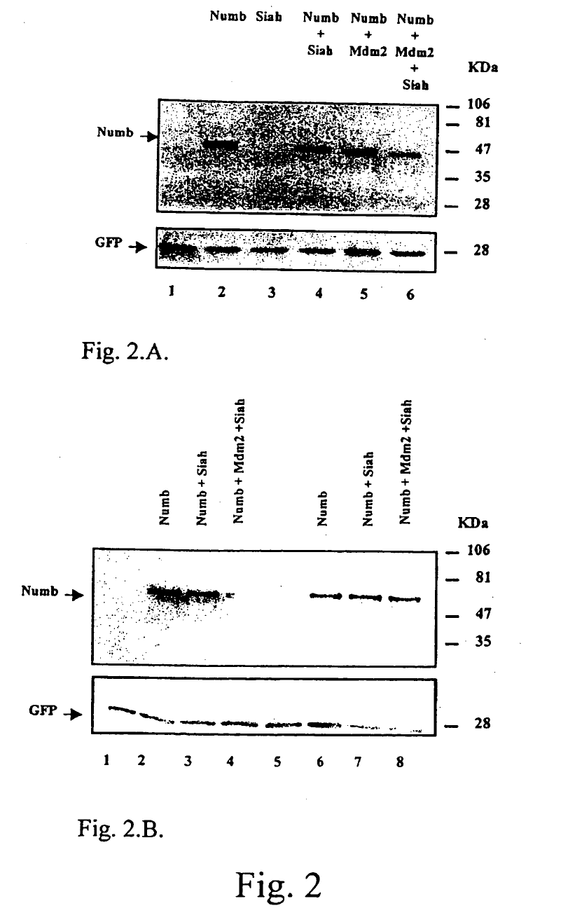

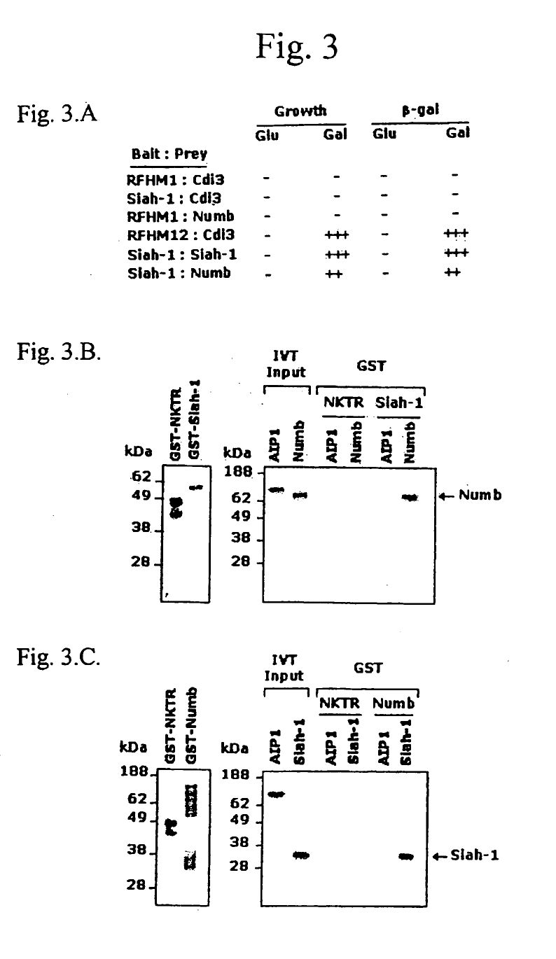

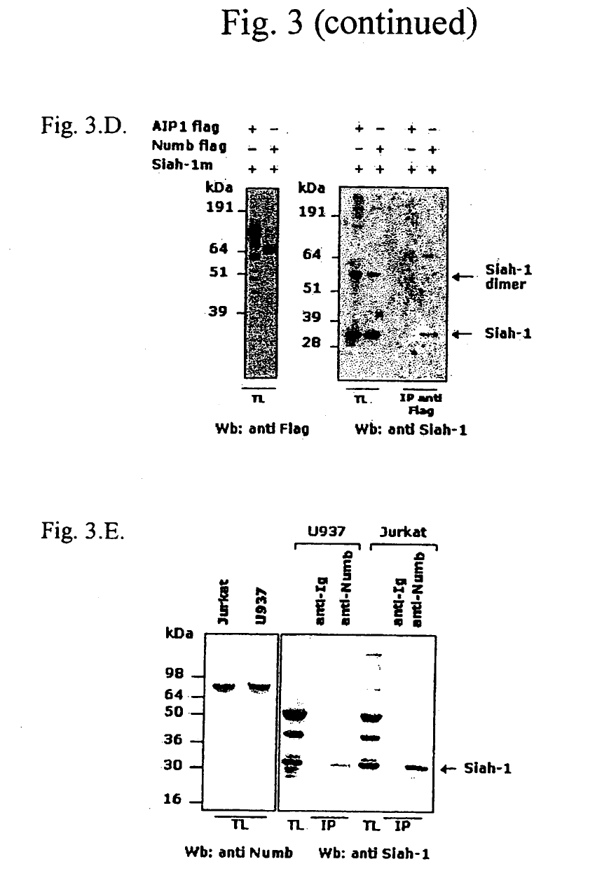

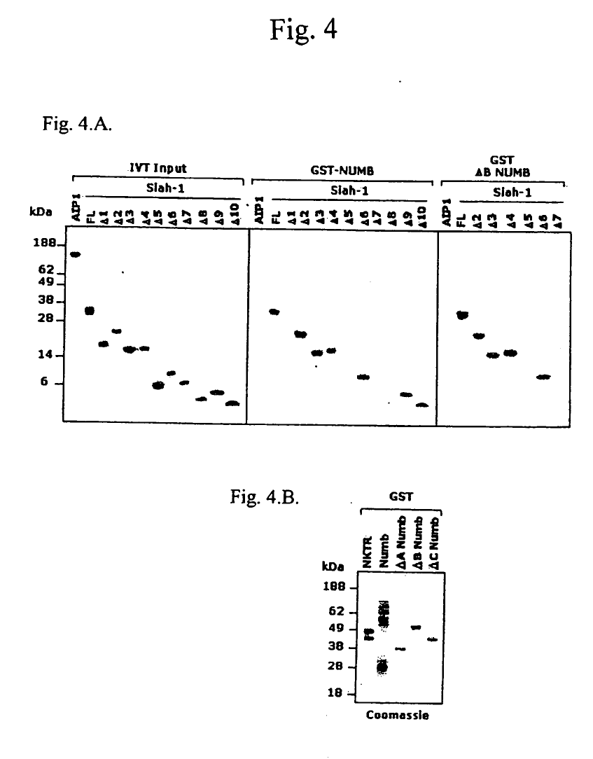

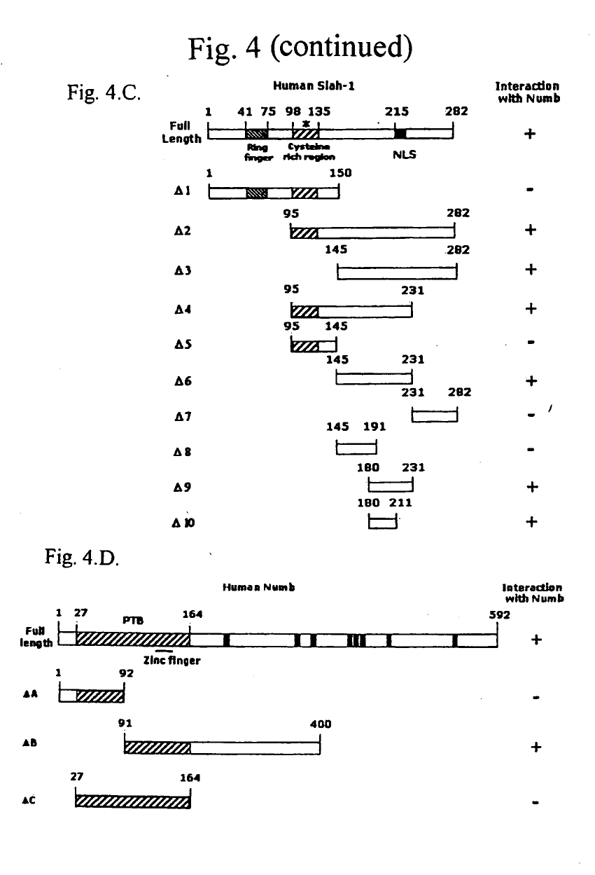

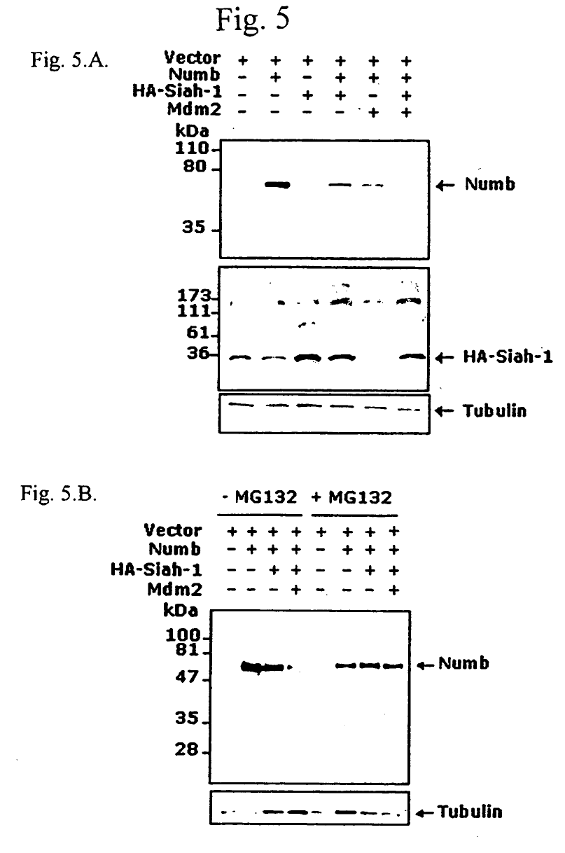

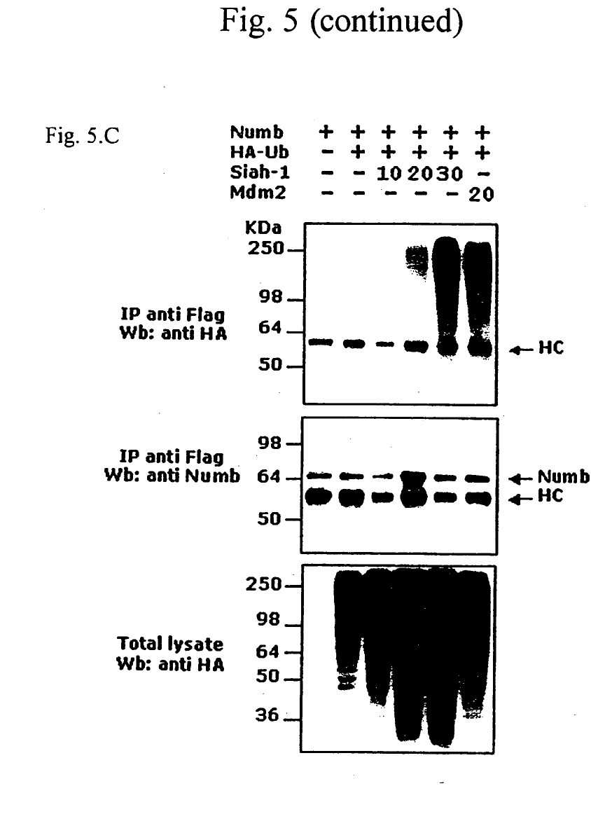

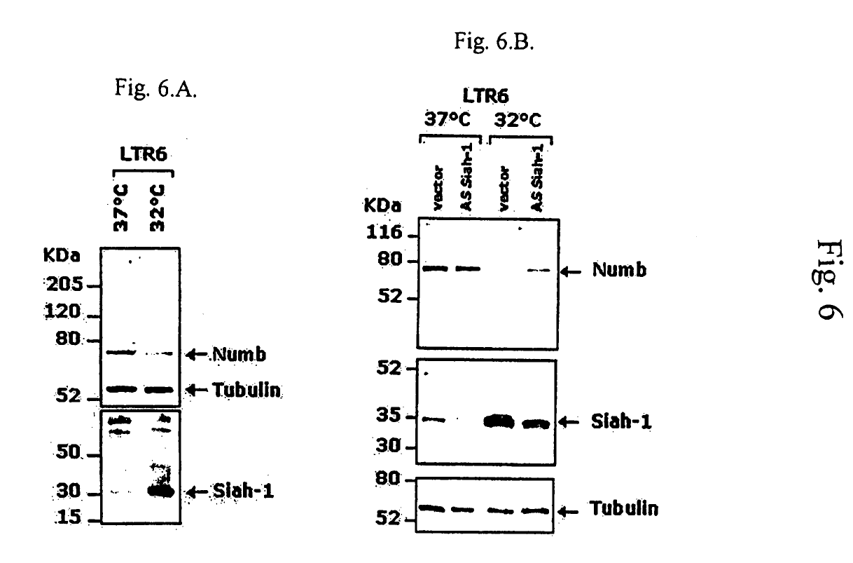

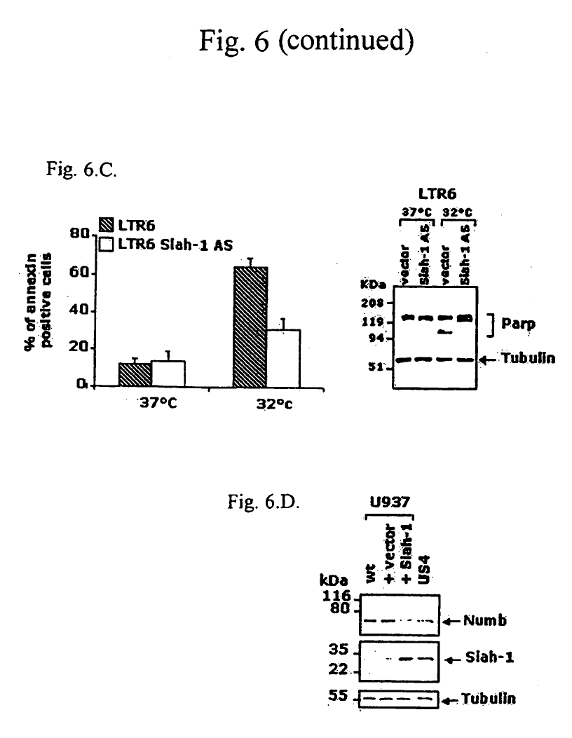

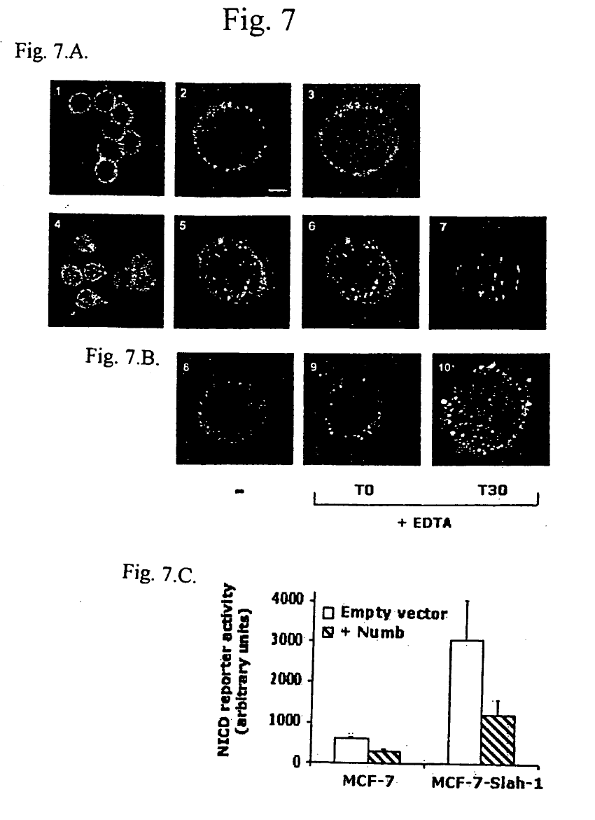

FIG. 2 clearly shows that the quantity of Numb protein decreases in the presence of Siah, in the same way as in the presence of Mdm2. These effects appear to be independent since the effects are summated when the Siah and Mdm2 proteins are both brought into contact with Numb.

FIG. 2B shows that the degradation of Numb by the Siah protein takes place by the ubiquitin proteasome pathway, since the degradation is inhibited by using an inhibitor of this pathway.

The results which are presented in the present application thus demonstrate the binding which exists between the Numb and Siah proteins and the fact that the Siah protein causes the Numb protein to be degraded by a pathway which is dependent on the ubiquitin proteasome.

It is also possible to use other techniques to demonstrate the binding between the Siah and Numb proteins, in particular the two-hybrid technique which is derived from the system developed by Finley and Brent (Annu Rev Genet 1997; 31: 663-704).

The bait protein can be cloned into the plasmid pEG202, which is known by the skilled person to be suitable for such an application (promoter 67-1511, lexa 1538-2227, ADH Ter 2209-2522, pBR remnants 2540-2889, 2μ ori 2890-4785, YSCNFLP 4923-5729, HIS3 7190-5699, TYIB 7243-7707, RAF_part 7635-7976, pBR backbone 7995-10166, bla 8131-8988).

The prey protein can be cloned into the plasmid pJG4-5, which is also well known to the skilled person (promoter GAL 1-528, fusion cassette 528-849, ADH Ter 867-1315, 2μ ori 1371-3365, TRP1 3365-4250, pUC backbone 4264-6422, Ap 4412-5274).

Use is also made of the reporter plasmid pSH18-34, which is also known to the skilled person. This plasmid is, in particular, available from Invitrogen, under reference number V611-20, and can also be obtained from the same supplier already transformed into the strain EGY48 (also termed RFY 231) (sole strain reference: C835-00, transformed with pSH18-34: C836-00).

The binding is demonstrated in the yeast strain RFY 231 (described in Finley Jr, et al, 1998, Proc Natl Acad Sci USA, 95, 14266-71). This yeast strain possesses the genotype (MATα trp1Δ::hisG his3 ura3-1 leu2::3Lexop-LEU2), and is derived from EGY48 (Guris et al., 1993, Cell, 75, 791-803).

The reporter gene was the LacZ gene.

The study is carried out on a medium containing galactose, and not containing leucine, and the presence of coloured colonies is examined on these dishes.

### Example 7

### Screening for Compounds which Interfere with the Siah-Numb Binding and/or the Quantity of Numb Protein

The assays described in Examples 1 to 6 are carried out while adding the compounds which it is desired to study, and a comparison is made with the results which are obtained when the compounds are not added.

1. Process for identifying a compound which inhibits the binding of Siah to Numb, characterized in that it exhibits the steps of:
a) bringing the said compound into contact with a mammalian cell which is expressing the Siah and Numb proteins;
b) identifying the increase in the quantity of Numb protein in the said mammalian cell as compared with a control cell with which the said compound has not been brought into contact.

2. Process for identifying a compound which inhibits the binding of Siah to Numb, which process exhibits the steps of:
a) bringing the said compound into contact with a system which enables the binding between Siah and Numb to be determined in vitro;
b) identifying the decrease in and/or inhibition of the binding between Siah and Numb.

3. Process for identifying a compound for increasing tumour suppression and/or cell death (apoptosis), characterized in that it exhibits the steps of:
a) bringing the said compound into contact with a mammalian cell which is expressing the Siah and Numb proteins;
b) identifying the decrease in the quantity of Numb protein in the said mammalian cell as compared with a control cell with which the said compound has not been brought into contact.

4. Process for identifying a compound for decreasing and/or inhibiting tumour suppression and/or cell death (apoptosis), which process exhibits the steps of:
a) bringing compounds into contact with a system which enables the binding between Siah and Numb to be determined in vitro;
b) identifying the compounds which induce a decrease in and/or inhibition of the binding between Siah and Numb;
c) bringing the compounds identified in step b) into contact with a mammalian cell which is expressing the Siah and Numb proteins;
d) identifying the increase in the quantity of Numb protein in the said mammalian cell as compared with a control cell with which the said compound has not been brought into contact.

5. Process for identifying a compound for increasing tumour suppression and/or cell death (apoptosis), characterized in that it exhibits the steps of:
a) identifying compounds which inhibit the binding between Siah and Numb, using a process in accordance with claim 2;
b) implementing a process in accordance with claim 3 on the said compounds identified in step a).

6. Process for identifying a region of Siah which is active in increasing tumour suppression and/or apoptosis, comprising the steps of:
a) bringing peptides derived from the Siah protein into contact with a mammalian cell which is expressing the Numb protein;
b) identifying the decrease in the quantity of Numb protein in the said mammalian cell by comparison with the quantity of Numb protein which is present in a control mammalian cell which is expressing the Siah and Numb proteins.

7. Process for identifying a region of Siah which is active in decreasing and/or inhibiting tumour suppression and/or apoptosis, comprising the steps of:
a) bringing peptides derived from the Siah protein into contact with a mammalian cell which is expressing the Numb protein;
b) identifying the increase in the quantity of Numb protein in the said mammalian cell by comparison with the quantity of Numb protein which is present in a control mammalian cell which is expressing the Siah and Numb proteins.

8. Process for identifying the regions of Siah which are involved in the binding with the Numb protein, comprising the steps of:
a) bringing peptides derived from the Siah protein into contact in a system which enables the binding between Siah and Numb to be determined in vitro;
b) identifying the peptides which bring about the decrease in the binding between Siah and Numb in the said system.

9. Process for identifying a product which is active in increasing tumour suppression and/or apoptosis, characterized in that it comprises the steps of:
a) implementing a process according to one of claims 3, 5 or 6,
b) modifying the product selected in step a), in particular by attaching residues to the chemical backbone,
c) testing the product modified in step b), in in vitro and/or in vivo processes, in models which are relevant to tumour suppression and/or apoptosis,
d) identifying the product which makes it possible to obtain a tumour suppression and/or apoptosis activity which is greater than the activity which is obtained in the case of the product selected in step a).

10. Process for identifying a product which is active in decreasing and/or inhibiting tumour suppression and/or apoptosis, characterized in that it comprises the steps of:
a) implementing a process according to one of claims 1, 2, 4 or 7,
b) modifying the product selected in step a), in particular by attaching residues to the chemical backbone,
c) testing the product modified in step b), in in vitro and/or in vivo processes, in models which are relevant to tumour suppression and/or apoptosis,
d) identifying the product which makes it possible to obtain a tumour suppression and/or apoptosis activity which is reduced as compared with the activity which is obtained in the case of the product selected in step a).

11. Process according to claim 1 or 4, characterized in that the said mammalian cell is transfected with at least one vector which is selected from a vector carrying a DNA fragment encoding Siah, a vector carrying a DNA fragment for Numb, and a vector carrying a DNA fragment for Siah and a DNA fragment encoding Numb.

12. Process according to claim 1 or 4, characterized in that the increase in the quantity of Numb protein in the said mammalian cell is identified by means of Western blotting.

13. Use of a compound, which is identified by a process according to one of claims 3, 5 or 9, and which is active in increasing tumour suppression and/or apoptosis, for preparing a drug which is intended for treating cancer.

14. Use of a compound, which is identified by a process according to one of claims 1, 2, 4 or 10, and which is active in decreasing and/or inhibiting tumour suppression and/or apoptosis, for preparing a drug which is intended for treating a neurodegenerative disease.

15. Peptide sequence which corresponds to a region of Siah which can be identified by a process according to one of claims 6 to 8.

16. Nucleotide sequence which encodes a peptide sequence according to claim 15.

17. Peptide sequence according to claim 15, or nucleotide sequence according to claim 16, as a drug.

18. Use of a peptide sequence according to claim 15, or of a nucleotide sequence according to claim 16, for preparing a drug.

19. Complex which consists of a Siah protein and a Numb protein.

20. Process for inhibiting the binding of Siah to Numb in a cell in vitro, comprising the step consisting in:
a) bringing the said cell into contact with a compound which has been identified by a process according to either claim 1 or 2.

21. Process for inhibiting a decrease in p53 in a cell in vitro, comprising the step consisting in:
a) bringing the said cell into contact with a compound which has been identified by a process according to one of claims 3, 5, 6 or 9.

22. Process for augmenting a decrease in the quantity of p53 in a cell in vitro, comprising the step consisting in:
a) bringing the said cell into contact with a compound which has been identified by a process according to one of claims 4, 7 or 10.
